(19) United States
(12) Patent Application Publication
(10) Pub. No.: US 2023/0064988 A1
(43) Pub. Date: Mar. 2, 2023

(54) SYSTEM AND METHOD OF VISIBILITY INCREASE FOR A CRYOCHAMBER

(71) Applicant: Hyper IP, LLC, Austin, TX (US)

(72) Inventors: Piotr Pietryga, Wroclaw (PL); Pawel Dorosz, Wroclaw (PL)

(21) Appl. No.: 17/463,863

(22) Filed: Sep. 1, 2021

(51) Int. Cl. A61F 7/00; F24F 3/14

(52) U.S. Cl. CPC A61F 7/0053; F24F 3/14

(57) ABSTRACT

The present invention relates to a visibility increase method for a cryochamber comprises pre-chamber equipped with air-drying system and heat exchanger ice removal system. The cryochamber can include an air dryer with at least one air dryer unit of any kind, and inlet and outlet ducts, which connect air dryer and pre-chamber. Both inlet and outlet of the air dryer are connected to the pre-chamber. Ice removal system can include at least one a pressurized gas source and for example an air knife, nozzle or orifice. Both systems improve user's field of view inside cryochamber, which greatly increases comfort of the user and for ice removal system, it additionally improves efficiency of the cryochamber.

### 1. FIELD OF THE INVENTION

The present invention relates to moisture content in a pre-chamber of a cryochamber.

### 2. DESCRIPTION OF THE RELATED ART

Cryochambers are used in whole-body cryotherapy, where user for the duration of the session stays inside an insulated space in very low temperature (−90° F. and below). Reoccurring problem is that due to moisture in air, mist forms for inside the cryochamber which greatly decreases visibility inside and as a result, comfort of the user being inside, as the depth perception can be disrupted, as well as limited presence of the light can also be detrimental to user's experience. Moreover, ice crystals can cover the surface of the heat exchanger inside the cryochamber taking active part in heat transfer between working fluid flowing inside heat exchanger and cooled air inside the cryochamber.

### SUMMARY OF THE INVENTION

In order to solve challenges and drawbacks mentioned above, a method of increasing visibility incudes drying a pre-chamber and/or using pneumatic to remove ice crystal formed on a heat exchanger inside the cryochamber. Equipping the pre-chamber with air-drying system can remove, minimize, or otherwise reduce moisture from the air, which can lead to increased visibility inside the cryochamber during sessions.

When people enter or exit the cryochamber door opens, moist in ambient air can flow inside the cryochamber, and that most can result in a significant amount of mist to form in the closed space. The mist can reduce a user's field of view during the session and their perception of depth, which can cause discomfort. By drying the air in a pre-chamber connected to the cryochamber, an amount of air moisture can be eliminated, minimized or otherwise reduced and, in turn, can eliminate, minimize, or otherwise reduce visibility loss during the session.

In some instances, air-drying system can air dryer with inlet and outlet ducts fluidically connecting the air dryer and the pre-chamber. For example, both the inlet and outlet of the air dryer are connected to the pre-chamber using the outlet and inlet ducts, respectively. In this configuration, the air-drying system can circulate air inside the pre-chamber constantly, periodically, intermittently, or a combination thereof while the cryochamber is operating.

As previously mentioned, moist in air in a cryochamber can result in ice, frost, or snow to form on a heat exchanger included in the interior of the cryochamber. To assist in its removal, an ice removal system can include at least one a pressurized gas connected to, for example an air knife that, when activated, can remove ice formed on the surface of the heat exchanger using pressured gas. The ice removal may include other devices that can direct pressured air at the heat exchanger in the cryochamber such as one or more nozzles, one or more orifices, one or more air knifes, or a combination thereof. The ice remove system may include other elements such as at least one pressure duct, at least one check valve, and/or others. This system works operate in response to an amount of ice crystals collected on the heat exchanger surface exceeding a predefined threshold. The high velocity gas flowing out of the outlet of the nozzle removes the ice as previously stated. In doing so, the visibility within the cryochamber can increase because the heat exchanger efficiency will increase and, in turn, remove more moisture from the air.

### BRIEF DESCRIPTION OF THE DRAWINGS

The structure, operating principle and effects of the present invention will be described in detail by way of various embodiments that are illustrated in the accompanying drawings.

FIG. 1 is a cross-sectional perspective view of the first embodiment of the present invention, with both cryochamber and pre-chamber door removed for clarity.

FIG. 2 is a perspective view of the backside of the first embodiment of the present invention with both cryochamber and pre-chamber door removed for clarity.

FIG. 3 is a cross-sectional perspective view of the second embodiment of the present invention with both cryochamber and pre-chamber door removed for clarity.

FIG. 4 is a perspective view of the backside of the second embodiment of the present invention with both cryochamber and pre-chamber door removed for clarity.

FIG. 5 is a detail view of the ice removal device of the second embodiment of the present invention.

FIG. 6 is a cross-sectional perspective view of the third embodiment of the present invention with both cryochamber and pre-chamber door removed for clarity.

FIG. 7 is a perspective view of the backside of the third embodiment of the present invention with both cryochamber and pre-chamber door removed for clarity.

FIG. 8 is a detail view of the ice removal device of the third embodiment of the present invention.

FIG. 9 is a flowchart illustrating an example method for increasing visibility in a cryochamber.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It will be acknowledged that when an element or layer is referred to as being “on,” “connected to” or “coupled to” another element or layer, it can be directly on connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise” and variations such as “comprises” or “comprising”, will be acknowledged to imply the inclusion of stated elements but not the exclusion of any other elements.

Embodiments of the disclosure provide a method of visibility increase for a cryochamber.

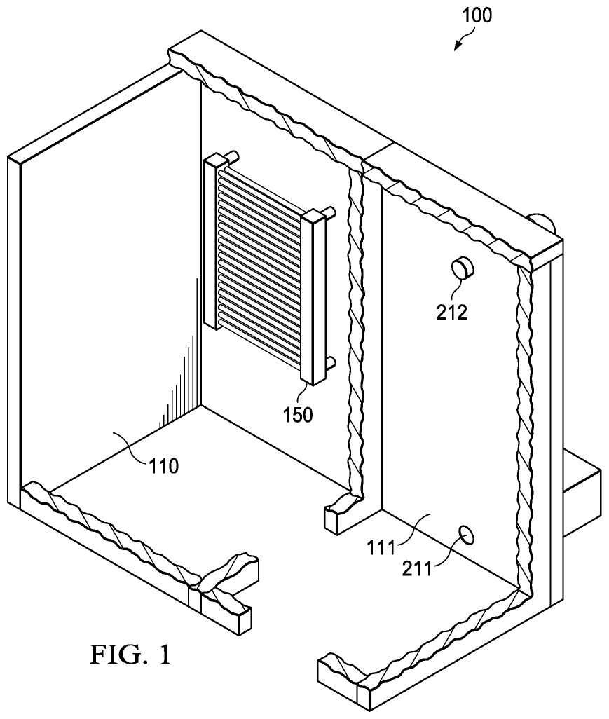

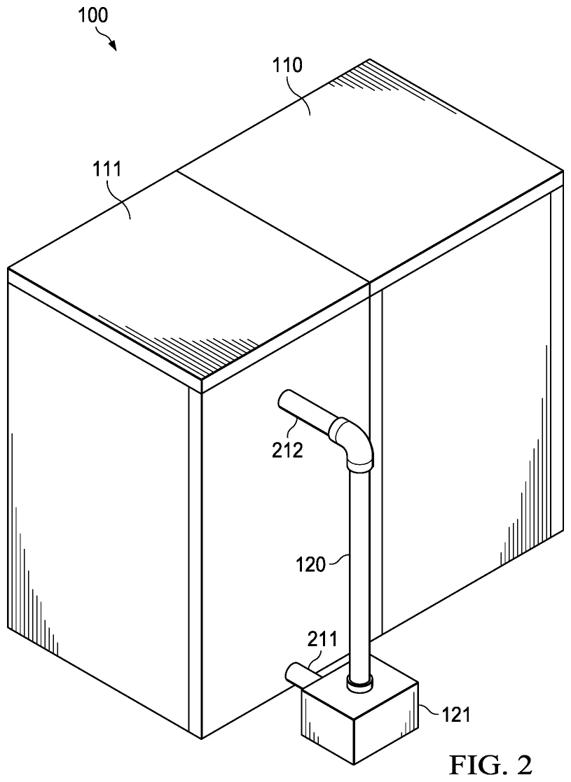

FIGS. 1 and 2 depict a cryochamber 100 connected with a pre-chamber 111 that removes moisture from air in the pre-chamber 111 prior to individuals entering or exiting the interior of the cryochamber 100. In some instance, the pre-chamber 111 dries air to eliminate, minimize, or otherwise reduce moisture entering the interior when individuals enter or exit the cryochamber 100.

As illustrated in FIG. 2, the pre-chamber 111 includes air-drying system 120 that receives air from the pre-chamber 111, dries the received air, and expels the dried air back into the pre-chamber 111. In the illustrated example, the air-drying system 120 includes an inlet duct 211 having an inlet connect to the pre-chamber 111 and outlet connect to an inlet of the drying unit 121. The drying unit 121 can be any type of air dryer that works in a constantly, periodically, intermittently, or a combination thereof, such as a desiccant air dryer. In this example, the air passes through a bed of desiccant material that removes moisture from the air through adsorption. Alternatively or in combination, the drying unit 121 can include a refrigerated air dryer that cools the incoming air to remove moisture, which can cause moisture from the air to condense inside the air dryer unit. Alternatively or in combination, the drying unit 121 can use other systems to remove moisture without departing from the scope of the disclosure. These methods can, in some instances, lower relative humidity inside the pre-chamber 111 from 40% to as low as 20%.

The drying unit 121 includes an inlet connected to an outlet of an inlet duct 211. The inlet of the inlet duct 211 can be positioned proximate the floor of the pre-chamber 111, such as, for example, 8 inches or less from the floor level. The inlet of the inlet duct 211 can be placed on any wall of the pre-chamber 111, such as near the outside door or across from the door between the cryochamber 100 and the pre-chamber 111. The air-drying unit 121 can be any type of air dryer suitable for this purpose without departing from the scope of the disclosure, and a plurality of air dryers 121 can be connected in parallel and/or serial connections in order to increase capacity of the air-drying system 120.

The drying unit 121 includes an outlet connected to an inlet of an outlet duct 212 and the outlet duct 212 includes an outlet connected to the pre-chamber 111. Placement of the outlet duct 212 can depend on the placement of the inlet duct 211 and outlet duct 212 such as the inlet duct 211 located directly above the outlet duct. In some instances, the inlet of the inlet duct 211 can be located proximate the ceiling of the pre-chamber 111 such as, for example, about 10 inches below the ceiling. Inlet duct 211 and outlet duct 212 can be made of any material and can be of any shape that provides small enough pressure drop for the air dryer 121 to operate. The air dryer 121 can have a capacity of 230 CFM (cubic feet per minute) of processed air, and pressure drop across the inlet duct 211 and outlet duct 212 can be in the range of 0.15-0.45 psi (pounds per square inch). The drying units 121 can be connect to multiple ducts at different heights and widths of the back wall 111 of, for example, the pre-chamber 111. Inlet duct 211 and outlet duct 212 can be connected to other sides of the pre-chamber 111 and/or different sides. Depending of the flow conditions and capacity required, different configurations can improve the efficiency of the air-drying system 120.

In some aspect of operations, the drying unit 121 pumps air from the pre-chamber 111 through the inlet duct 211 and dries are using, for example, a heater, absorbent, refrigerator, or other mechanism, or a combination thereof without departing from the scope of the disclosure. The drying unit 121 pumps the dried air into pre-chamber 111 through the outlet duct 212.

When a door of the pre-chamber 111 opens it is exposed to the ambient (e.g., at the beginning or ending of the cryotherapy session). While the door is open, air (e.g., ambient air) carrying moisture will flow inside the pre-chamber 111. As mentioned previously, the air-drying system 120 works intermittently, periodically, or constantly while the cryochamber 100 operates and removes moisture from the air. In doing so, the amount of moisture getting inside the cryochamber 100 is eliminated, minimized, or otherwise reduced when the one or more outside doors between interior and exterior of the pre-chamber 111 is closed and when one or more doors between the pre-chamber 111 and the cryochamber 100 are opened, so the user can begin their session.

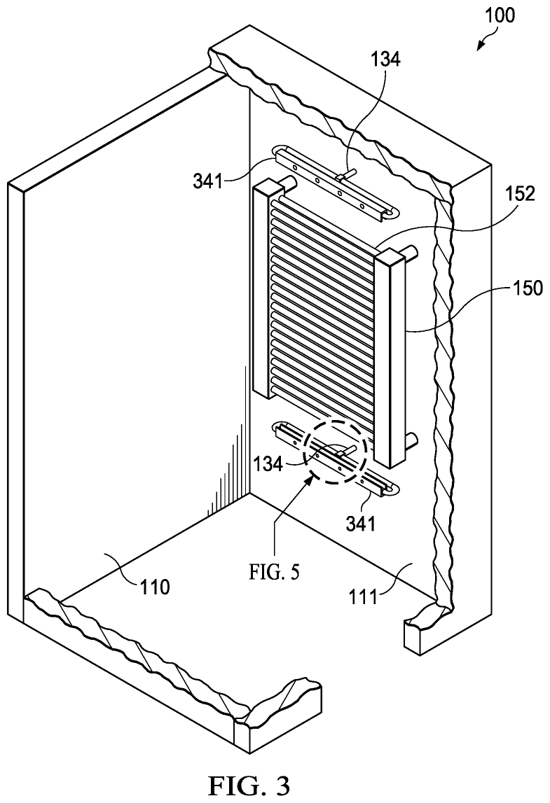

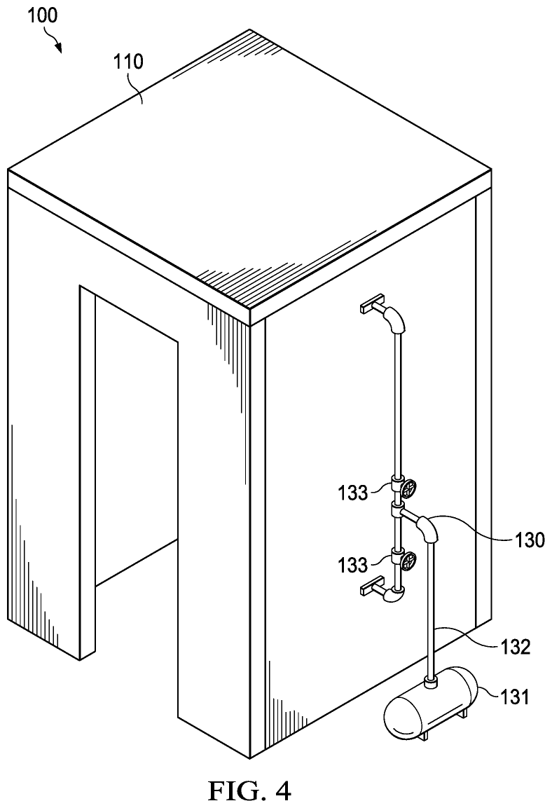

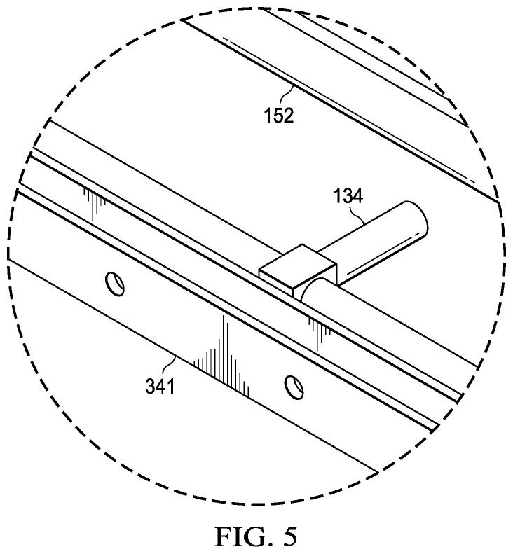

FIGS. 3 to 5 depict an ice removal device 130 for removing ice from the heat exchanger 150 in accordance with some implementations of the present disclosure. For example, the heat exchanger ice removal device 130 can include at least one pressurized gas source 131 fluidically connected to a plurality of air knives 341 through a pressure duct 132. When pressurized, the plurality of air knives 341 releases high-pressure gas across a surface of the heat exchanger 150 exposed to an interior of the cryochamber 100. For example, the plurality of air knives 341 may include, for example, one or more openings and include a beveled or tapered edge to increase the pressure of released gas. To increase the pressure of the gas, the openings of the air knives 341 can have a smaller diameter than cross-sectional are of the passage defined by the pressure duct 132. The surface of the air knife outlet should be at least two times lower than cross-sectional area of the pressure duct 132. These openings are directed across the surface of the heat exchanger 150 to remove ice. For example, the openings may form an acute angle with the surface of the heat exchanger 150 in a range of 20° to 85°. The air knives can be used to create strong air jets—they operate like nozzles and very high gas velocities (Ma˜0.2) can be achieved. Outlet channels of the air knives are generally rectangular (width less than ⅜ inches, length of the channel can be for example 40 inches), so they are useful in cases when we want to blow the air on a long object (for example, a pipe). The outlet of the channels can be rectangular, circular, elliptical, other shapes, or a combination thereof without departing from the scope of the disclosure. The two check valves 133 can assist in increasing kinetic energy of the pressurized gas 134.

With the cryochamber door opening periodically, ambient air enters the cryochamber 150. The surface of the heat exchanger 150 is below freezing, and, when moist in the ambient contacts the surface, ice crystals can form on the surface of the heat exchanger 150. Alternatively or in combination, ice suspended in the air inside the cryochamber 150 can adhere to the surface of the heat exchanger 150. Ice formation on the surface of the heat exchanger 150, such as through these mechanisms, can decrease the heat transfer efficiency of the heat exchanger 150, which in turn, can decrease the therapeutic effects of the cryochamber 100. In other words, the surface temperature of the heat exchanger 150 can be significantly lower that temperature inside the cryochamber 100. For example, this may occur when the cryochamber 100 begins operation at the start of the working day, when the air inside the cryochamber 100 is at the ambient temperature of 68° F. and the surface of the heat exchanger 150 is at −90° F. and below, or other situations. During normal operation, the temperature difference between the air inside the cryochamber 100 and heat transfer 150 surface can be about 40° F. While the ice collecting on the surface of the heat exchanger 150 can decrease the amount of moisture in the air, the ice on the surface can act as thermal resistance and decrease the efficiency of the heat exchanger 150 as the collected ice layer acts a thermal resistance. Depending of ambient conditions, the amount of users per session and number of sessions, heat transfer efficiency can decrease as much as 40%.

To counter the impact of the ice layer, the ice removal system 130 can be activated in response to, for example, a period of time, the temperature of the cryochamber 100 increase above a threshold, temperature different between the heat exchanger 150 and the interior of the cryochamber 100, a number of treatments, a thickness of the ice layer exceeding a threshold, a combination thereof, or other parameters. For example, the ice removal system 130 can be activated after every 5 sessions or every half an hour. In response, the plurality of air knives 341 direct pressure gas across the surface of the heat exchanger 150 to eliminate, minimize, or otherwise reduce the ice layer. Up to 90% of the residual ice can be removed from heat exchanger 150 surface with every activation of the system. Removing at least a portion of the ice layer can regenerate the ability of heat exchanger 150 to remove ice crystals from the air and increase its heat exchange efficiency. Pressurized gas tank of any type or multiple pressurized gas tanks of any type can be connected in parallel or serial connection or combination thereof. The pressure source 131 can include any compressor of any type or multiple compressors of any type connected in parallel or serial connection or a combination thereof without departing from the scope of the disclosure.

In some implementations, the check valves 133 can turn the cryochamber 100 on or off. For example, the check valves 133 can operate manually and/or automatically, and can close separately and/or together.

The kinetic energy of the pressurized gas can increase using, for example, a nozzle, an orifice, and/or other elements that restrict flow of the gas. Pressure of the gas, depending on the kind of element used, can be in the range of 30-300 psi. Multiple elements can be connected in parallel or serial connection or both can be used, as well as the elements can be installed in any location near the heat exchanger, such as below, in the front, as long as pressurized gas can flow between the heat exchanger tubes. In this embodiment, outlets of the orifices are directly below the heat exchanger tubes 152. Outlets of the orifices can also be placed on the either side of the heat exchanger surface 150, so the gas flows horizontally and sufficient clearance is also provided for this configuration.

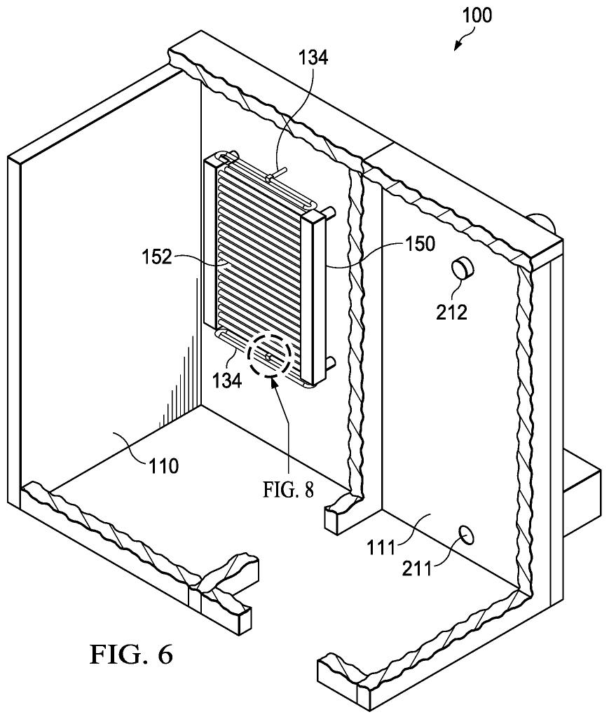

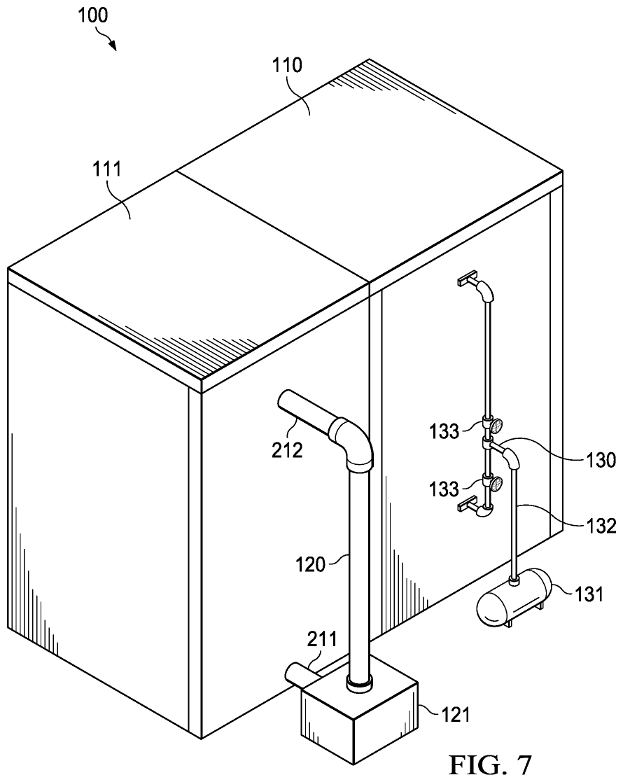

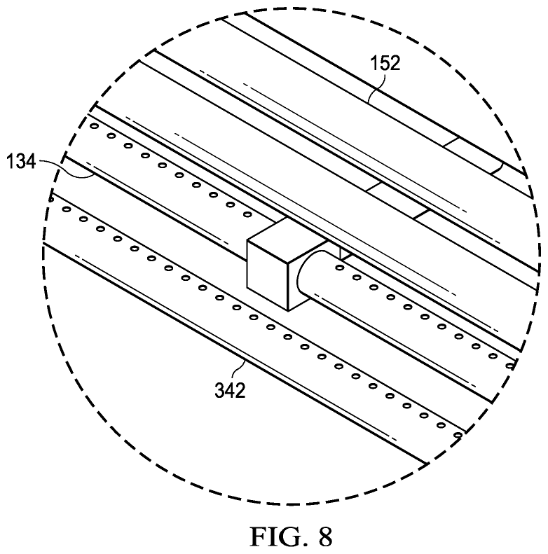

FIGS. 6 to 8 illustrates a combination of the air-drying system 120 and the ice removal system 130 in accordance with some implementations of the present disclosure. As illustrated, the air-drying system 120 eliminates, minimizes, or otherwise reduces moisture in the interior of the cryochamber 100 and the ice removal system 130 eliminates, minimizes, or otherwise reduces ice from the surface of the heat exchanger 150. In other words, this embodiment combines two previously presented embodiments, which can improve the proposed visibility.

In this embodiment, air-drying system 120 works together with ice removal system 130. Ice removal system is equipped with plurality of orifices 342 that provide different velocity distribution compared to air knives 341, as shown in the second embodiment, as well as their relative placement to the heat exchanger 150 surface is set up so the high velocity gas stream flows between the heat exchanger tubes 152.

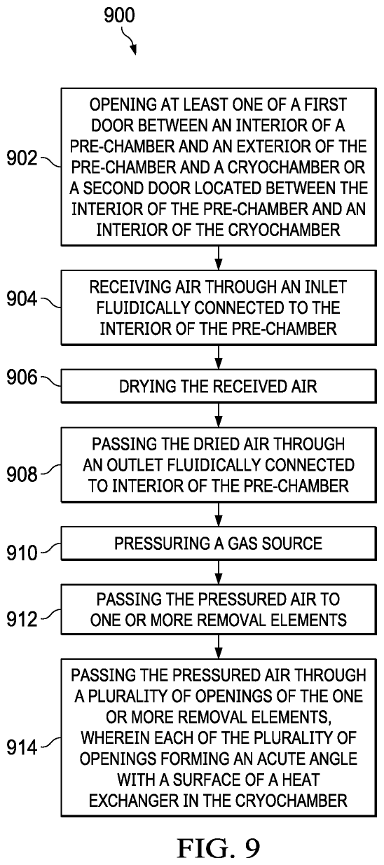

FIG. 9 illustrates a flowchart for drying air and reducing an ice layer on a heat exchanger in a cryochamber.

What is claimed is:

1. A system for cryogenic treatments, comprising:
a cryochamber comprising a heat exchanger with a surface exposed to an interior of the cryochamber; and
a pre-chamber connected to the cryochamber and comprising:
a first door between the interior of the pre-chamber and an exterior of the pre-chamber and the cryochamber;
a second door located between the interior of the pre-chamber and an interior of the cryochamber; and
an air drying having an inlet fluidically connected to the interior of the pre-chamber and an outlet fluidically connected to interior of the pre-chamber.

2. The system of claim 1, wherein the air dryer comprises at least one of a desiccant air dryer or a refrigerated air dryer.

3. The system of claim 1, the pre-chamber further comprises:
an inlet duct having an inlet connected to the interior of the pre-chamber and an outlet connected to the inlet of the air dryer; and
an outlet duct having an inlet connected to the outlet of the dryer and an outlet connected to the interior of the pre-chamber.

4. The system of claim 3, wherein a center of the outlet of the outlet duct is 8″ or less from a floor of the pre-chamber.

5. The system of claim 3, wherein a center of the inlet of the inlet duct is 10″ or less from a ceiling of the pre-chamber.

6. The system of claim 1, the cryochamber further comprising:
a pressure gas source;
a conduit having an inlet connected to an outlet of the pressured gas and outlet connected to one or more removal elements, wherein the conduit defines passages having a cross-sectional area; and
the one or more removal elements defining a plurality of opening and each of the plurality of openings forming an acute angle with the surface of the heat exchanger, wherein an area of each of the plurality of openings is less than the cross-sectional area.

7. The system of claim 6, wherein the one or more removal elements comprises at least one of an air knife, a nozzle, or a check valve.

8. A method, comprising:
opening at least one of a first door between an interior of a pre-chamber and an exterior of the pre-chamber and a cryochamber or a second door located between the interior of the pre-chamber and an interior of the cryochamber; and
receiving air through an inlet fluidically connected to the interior of the pre-chamber;
drying the received air; and
passing the dried air through an outlet fluidically connected to interior of the pre-chamber.

9. The method of claim 8, wherein the received air is dried using at least one of a desiccant air dryer or a refrigerated air dryer.

10. The method of claim 8, wherein the inlet receives the air through an outlet of an inlet duct having an inlet connected to the interior of the pre-chamber; and
wherein the outlet passes the dried air through an inlet of an outlet duct and through an outlet of the outlet duct connected to the interior of the pre-chamber.

11. The method of claim 10, wherein a center of the outlet of the outlet duct is 8 inches or less from a floor of the pre-chamber.

12. The method of claim 10, wherein a center of the inlet of the inlet duct is 10 inches or less from a ceiling of the pre-chamber.

13. The method of claim 8, further comprising:
pressuring a gas source;
passing the pressured air to one or more removal elements; and
passing the pressured air through a plurality of opening or the one or more removal elements, wherein each of the plurality of openings forming an acute angle with a surface of a heat exchanger in the cryochamber.

14. The method of claim 13, wherein the one or more removal elements comprises at least one of an air knife, a nozzle, or a check valve.

15. A computer program product encoded on a non-transitory medium, the product comprising computer readable instructions for causing one or more processors to perform operations comprising:
opening at least one of a first door between an interior of a pre-chamber and an exterior of the pre-chamber and a cryochamber or a second door located between the interior of the pre-chamber and an interior of the cryochamber; and
receiving air through an inlet fluidically connected to the interior of the pre-chamber;
drying the received air; and
passing the dried air through an outlet fluidically connected to interior of the pre-chamber.

16. The computer program product of claim 15, the instructions further causing the one or more processors to perform operations comprising:
pressuring a gas source;
passing the pressured air to one or more removal elements; and
passing the pressured air through a plurality of opening or the one or more removal elements, wherein each of the plurality of openings forming an acute angle with a surface of a heat exchanger in the cryochamber.

17. The computer program product of claim 15, wherein the received air is dried using at least one of a desiccant air dryer or a refrigerated air dryer.

18. The computer program product of claim 16, wherein the one or more removal elements comprises at least one of an air knife, a nozzle, or a check valve.
